(19) United States
(12) Patent Application Publication
(10) Pub. No.: US 2006/0167329 A1
(43) Pub. Date: Jul. 27, 2006

(54) Reactor

(76) Inventor: Matthias Grahl, Munchen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/316,320

(22) Filed: Dec. 23, 2005

(30) Foreign Application Priority Data
Jan. 21, 2005 (DE) 102005002975.2

(51) Int. Cl. C07C 7/12; B01J 8/04; B01J 10/00

(52) U.S. Cl. 585/821; 422/193; 422/192; 422/211

(57) ABSTRACT

A reactor for chemical reactions and/or adsorptive separation processes comprise having a jacket (1); a central hollow column (2,2′); two essentially annular beds (6, 6′, 7, 7′, 7″) comprise an outer bed (6, 6′) consisting essentially of a chemically and/or adsorptively active material (active bed) and an inner bed (7, 7′, 7″) consisting of a material that is inert with respect to the chemical reaction that is to be carried out in the reactor (inert bed); the beds (6, 6′, 7, 7′, 7″) being separated from one another by preferably a gas-permeable and/or liquid-permeable barrier or wall; a gas feed and gas exhaust area (4) between the jacket (1) and the active bed (6, 6′) and a gas feed and gas exhaust area associated with the central column (2, 2′). The inert bed (7, 7′, 7″) at least partially and preferably completely fills a space between the central column (2, 2′) and the active bed (6, 6′).

The invention relates to a reactor for chemical reactions and/or adsorptive separation processes,
a) having a jacket,
b) having two essentially annular beds,
c) one bed consisting of a chemically and/or adsorptively active material (active bed) and one bed consisting of a material that is inert with respect to the chemical reaction that is to be carried out in the reactor (inert bed),
d) the beds being separated from one another by means of a gas-permeable and/or liquid-permeable barrier,
e) having a gas feed and gas exhaust area between the jacket and active bed and
f) having a gas feed and gas exhaust area in the form of a central column.

The expression “active bed” is defined as any type of bed that is formed from one or more different materials, the material or materials reacting chemically and/or adsorptively actively with respect to the medium that is routed over it or through it—whether gaseous or liquid.

The expression “inert bed” is defined as any type of bed that is formed from one or more different materials, the material or materials reacting neither chemically nor adsorptively with respect to the medium that is routed over it or through it—whether gaseous or liquid.

European Patent Application 0 759 320 discloses a generic reactor for chemical reactions that is used especially for adsorptive separation processes.

There is a further application for generic reactors. They can be used for the most varied reactions between a gas or a liquid and an active material. The active material can be, for example, an adsorbent or a catalyst.

During the reaction phase, a reaction gas is routed through a bed comprising the active material, for example, to a space between the reactor jacket and the outer porous barrier of the bed, and is withdrawn from the inner porous barrier of the bed. Of course, the flow direction can also run in reverse; in this case, the reaction gas is first supplied through the inner porous barrier, then after passing through the bed it travels into the space between the reactor jacket and outer porous barrier and is withdrawn from this space.

In the case of using such a reactor for an adsorptive separation process, it can be taken into account that the adsorption capacity of the active material, therefore of the adsorbent, decreases as the time interval of charging increases. Therefore, the adsorbent must be regenerated at regular intervals. In this connection, during the regeneration phase, the pressure is reduced and/or a regeneration gas that has a different chemical composition and/or a different thermodynamic state relative to the reaction or feed gas that is to be cleaned is routed through the bed of active material.

In generic reactors, the gas-permeable and/or liquid-permeable area between the inner and outer barrier ends underneath the top edge of the bed. The same applies to beds through which flow takes place horizontally. The reactor is supplied with the active material, in general by way of a manhole that is located in the upper area of the reactor. Generally, the bed extends into this manhole.

Generic reactors or absorbers require special measures to achieve uniform incident flow of the bed through which flow takes place horizontally—optionally in a continuously changing direction—since the bed itself produces relatively little pressure drop and during individual process steps very high velocities—up to 100 m/s—can occur. In order to achieve uniform incident flow into the bed, European Patent Application 0 870 538 proposes a reactor of radial design that has a deflection arrangement in the form of a cylindrical deflection plate. In this connection, the deflection arrangement can be provided either between the outer cladding and the bed or between the bed and the central axis of rotation.

In the aforementioned European Patent Application 0 759 320, a free volume on at least one vertical side of the bed is proposed to make the incident flow uniform i.e. to reduce the variations in local flow rate across the adsorbent; this free volume is divided by a wall into a first subvolume in the vicinity of the bed and a second subvolume. In addition, in the interior of the bed, an annular layer of divided material, for example aluminum balls, is proposed.

A poor or non-uniform distribution of the medium that is supplied to the active bed in adsorptive processes leads to premature breakthrough of one or more undesirable components in certain areas of the bed and thus to contamination of the product flow.

This is especially critical in vacuum cyclic pressure adsorption processes in which very high flow velocities often occur. Since in these processes the flow direction reverses in rapid sequence, a uniform distribution—therefore uniform incident flow into the active bed and uniform outflow out of the active bed—is necessary in both flow directions over a large range of velocities.

This problem is solved by means of relatively complicated tank internals as they are proposed in, for example, European Patent Application 0 870 538, but makes the reactor much more expensive. The reactor design disclosed in European Patent Application 0 759 320 does avoid this disadvantage, but it has been shown that with this approach, uniform incident flow and outflow cannot always be ensured. This applies especially when the axial symmetry of the reactor or adsorber is disrupted by production tolerances, inhomogeneities of the bulk density or even the structural configuration of the head area. In such cases, in the part of the inner gas volume that faces the bed, unwanted vertical velocity components can occur that prevent uniform use of the entire bed.

Object of this invention is to provide an improved generic reactor, especially one that reduces or avoids one or more of the aforementioned disadvantages.

Upon further study of the specification and appended claims, further objects and advantages of the invention will become apparent.

Thus, a generic reactor is proposed that is characterized in that the inert bed at least partially fills the space that remains between the central column and the active bed.

According to the invention, in the area between the inner free gas volume and the active bed, there is therefore now a layer of inert material that at least partially fills this area.

It has been found that with the reactor design according to the invention, even at high flow velocities and for rapidly changing flow directions, a relatively good uniform distribution can be achieved.

Other special embodiments of the reactor according to the invention include one or more of the following features:
the inert bed extends over at least 10% of the height of the inner entry or exit area of the active bed, preferably over the entire height of the inner entry or exit area of the active bed,
the inert bed is formed from metallic and/or ceramic packings, preferably from Pall or Raschig rings,
the Pall and/or Raschig rings preferably have a diameter between 10 and 50 mm,
the layer thickness of the inert bed is between 5 and 50%, preferably between 10 and 20%, of the layer thickness of the active bed,
the gas-permeable and/or liquid-permeable wall that separates the beds is a perforated sheet,
there is no gas-permeable and/or liquid-permeable partition between the active bed and the inert bed and
in addition to the inert bed that is located between the central column and the active bed, there is another inert bed that surrounds the active bed.

### BRIEF DESCRIPTION OF DRAWINGS

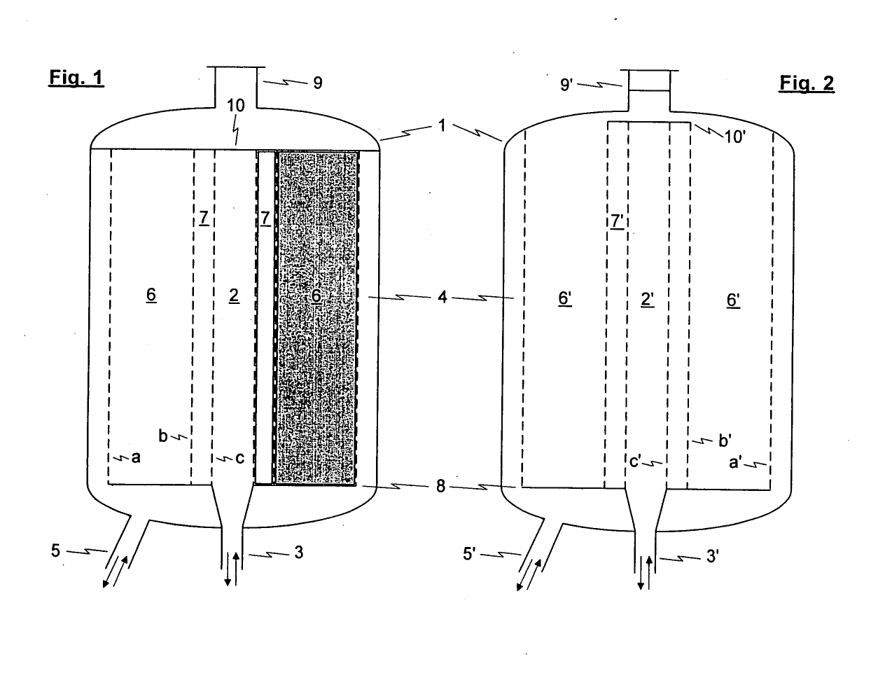

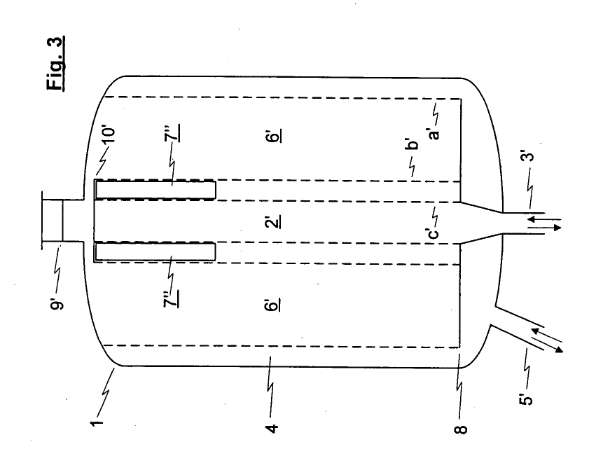

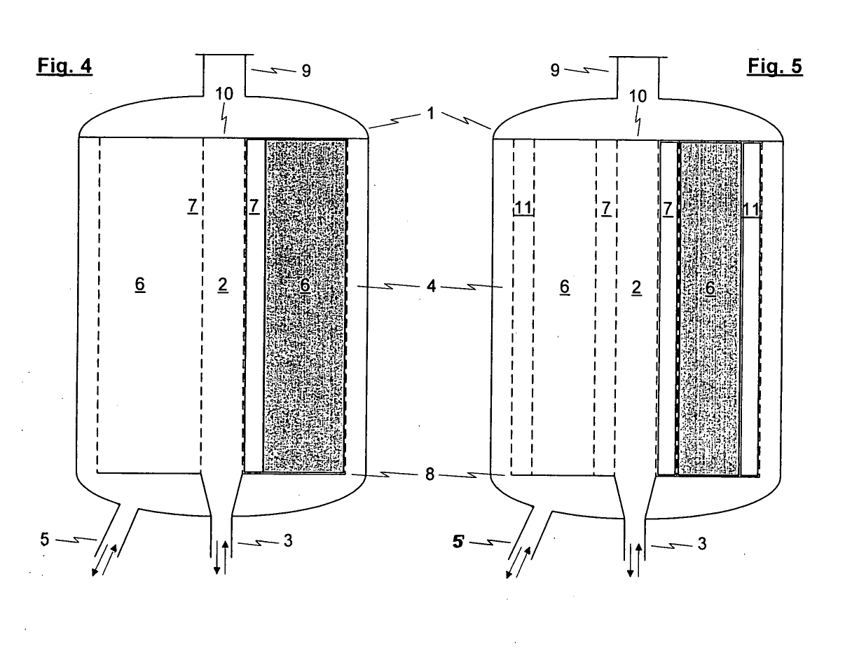

The reactor according to the invention and other embodiments thereof are explained in more detail below with reference to the schematically illustrated preferred embodiments shown in FIGS. 1, 2, 3, 4, and 5 wherein the prime numbers refer to FIGS. 2 and 3 and the double prime numbers refer to FIG. 3. Each of FIGS. 1, 2, 3, 4, and 5 show a lateral cross-section of embodiments of the reactor 1, the variant shown in FIG. 2 differing from the variant shown in FIG. 1 in that the active bed 6′ extends into the head area of the reactor 1, by which the radial symmetry of the bed is disrupted. The embodiment of the reactor 1 according to the invention that is shown in FIG. 3 is characterized in that the inert bed 7″ is located only in the head area of the reactor 1. FIGS. 4 and 5 illustrate still further embodiments, as described below.

### DETAILED DESCRIPTION OF DRAWINGS

Let it be emphasized that for the sake of simplicity, the figures are only schematic; in particular, the ratios between the external dimensions of the reactors 1 and the material thicknesses do not correspond to actual dimensions.

The illustrated reactors each have a chemically active bed 6/6′—hereinafter called the active bed—and a chemically inactive bed 7/7′/7″—hereinafter called the inert bed; the gaseous or liquid medium that is to be treated flows through both essentially horizontally. For this reason, for example, the liquid or gaseous medium is supplied to the inner central column 2/2′ by way of the gas supply and exhaust line 3. After passing horizontally through the beds 7/7′/7″ and 6/6′, the medium is withdrawn from the reactor 1 by way of the outer space 4 and the gas feed or exhaust line 5/5′. If at this point the flow direction is reversed, the medium enters the bed 6/6′ and 7/7′/7″ by way of the above-described line 5/5′ and the outer gap or space 4 and is then withdrawn from the reactor 1 according to the invention by way of the central column 2/2′ and the line 3/3′. The above-described flow guidance is shown schematically by means of the arrows drawn into the line 3/3′ and 5/5′.

The central column 2/2′ according to the prior art is made such that a first rough distribution of the gas flow over the length of the column is ensured. To do this, the free area of the lateral openings may not be too large. In general, it is conventional to dimension the free area such that the ratio between the free area and the column cross-section is not greater than 1.

The active bed 6/6′ is bordered in each case by one outer a/a′ and one inner b/b′ gas-permeable and/or liquid-permeable porous barrier or wall and on its bottom by a base 8. In the figures, the upper attachment 10 of the first bed 6/6′ and of the second bed 7/7′/7″ is shown only in schematic form. In practice, there are means known to one skilled in the art for compressing the beds 6/6′ and 7/7′/7″; they are, for example, made as a membrane and/or as a tank that can be filled with gas. Such means for compressing beds are useful to suppress the unwanted dust formation caused by abrasion and to compact the beds 6/6′ and 7/7′/7″ themselves. Furthermore, these means for compressing the beds are used to effectively prevent possible formations of unwanted bypass flows in the head area of the beds.

As already mentioned, the embodiments of the reactor according to the invention that are shown in FIGS. 1 and 2 and 3 differ in that in the case of FIGS. 2 and 3, the active bed 6′ extends into the head area of the reactor 1, more exactly into a manhole 9′ or the like that is normally provided. FIGS. 2 and 3 do not show the attachment of the bed surface that can optionally be provided, for example, by means of suitable flexible and compressible mats or inflatable tanks.

The inert bed 7/7′/7″ consists essentially or consists of a material that is inert relative to the chemical and/or adsorptive reaction that is to be carried out in the reactor. The inert bed extends in the embodiments of the reactor 1 according to the invention that are shown in FIGS. 1 and 2 over the entire height of the inner entry and exit area of the active bed 6/6′. It is separated from the latter by a gas-permeable and/or liquid-permeable wall b/b′. In the embodiments of the reactor 1 according to the invention that are shown in FIG. 3, however, the inert bed 7″ simply extends roughly over the uppermost third of the inner entry or exit area of the active bed 6. A host of other embodiments of the reactor 1 according to the invention are conceivable in which variants are selected that are different with respect to the height of the inert bed 7″.

The embodiment of the reactor 1 according to the invention that is shown in FIG. 3 has the advantage that with it maldistributions caused by the asymmetrical configuration of the head area can be specifically minimized.

Since the inert bed 7/7′/7″—in contrast to the known prior art—now directly borders the active bed 6/6′ and the central column 2/2′, a highly efficient uniform distribution in both flow directions is achieved. In the case of the flow direction “inside to outside,” a maldistribution that has remained after emerging from the central column 2/2′ is effectively compensated, in particular unwanted vertical velocity components are minimized. For the reverse flow direction, the outflow from the bed is made uniform; thus, for example, the maldistributions caused by a non-uniform bulk density can be attenuated and local breakthroughs of unwanted components can be minimized.

Let it be emphasized again that other internals or measures within the central column 2/2′ and/or the outer gap 4 as are suggested by the prior art are not necessary. Thus, embodiments of this invention are devoid of internals or measures within the central column and/or the outer gap 4.

The object of making the incident flow into the (first) bed uniform—can thus be achieved without complicated reactor internals or measures that are complex and thus expensive to produce. The materials used for the inert bed are comparatively economical.

Preferably, the inert bed 7/7′/7″ consists of metallic and/or ceramic packings, especially Pall and/or Raschig rings being used; here, the Pall and/or Raschig rings used preferably have a diameter of between 10 and 50 mm.

In developing the reactor according to the invention, it is proposed that the layer thickness of the inert bed 7/7′/7″ be between 5 and 50%, preferably between 10 and 20% of the layer thickness of the active bed 6/6′.

The gas-permeable and/or liquid-permeable walls a, a′, b, b′, c and c′ shown in FIGS. 1, 2 and 3 are preferably formed by perforated sheets.

It is furthermore conceivable that according to another advantageous configuration of the reactor according to the invention, there is no gas-permeable and/or liquid-permeable partition between the active bed 6/6′ and the inert bed 7/7′/7″ as illustrated in FIG. 4. This is only possible, however, when during the filling of the reactor according to the invention, suitable precautions are taken for separate filling of the two beds, for example in the form of a flexible ring that is drawn upward as the bed surfaces rise. Moreover, the materials for the active and inert bed must be chosen such that they cannot intermix during operation.

According to another configuration of the reactor according to FIG. 5, in addition to the inert bed 7/7′/7″ that is located between the central column 2/2′ and the active bed 6/6′, there can be another inert bed 11 that surrounds the active bed 6/6′ on the outside. This configuration of the reactor according to the invention has the advantage that the gas distribution can be improved on the outside as well.

The reactor design according to the invention is suited, as already mentioned, for adsorptive separation processes. In particular, they are especially well suited for adsorptive separation of nitrogen from a nitrogen-containing gas mixture, especially from air, or for adsorptive separation of carbon dioxide from a carbon dioxide-containing gas mixture, for example from a reducing exhaust gas.

Fundamentally, the reactors according to the invention can be advantageously used for any adsorptive separation of one or more components from a multi-component (gas) mixture or for separation of a multicomponent (gas) mixture.

The entire disclosures of all applications, patents and publications, cited herein and of corresponding German application No. 102005002975.2, filed Jan. 21, 2005 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A reactor for chemical reactions and/or adsorptive separation processes, comprising:
a) a jacket (1),
b) two essentially annular beds (6, 6′, 7, 7′, 7″),
c) one bed (6, 6′) consisting essentially of a chemically and/or adsorptively active material (active bed) and one bed (7, 7′, 7″) consisting essentially of a material that is inert with respect to the chemical reaction that is to be carried out in the reactor (inert bed), said one bed (6,6′) surrounding said one bed (7,7′,7″),
d) the beds (6, 6′, 7, 7′, 7″) being separated from one another by means of a gas-permeable and/or liquid-permeable barrier,
e) gas feed/gas exhaust space (4) between the jacket (1) and the active bed (6, 6′) or optionally between the jacket (1) and an inert bed surrounding the active bed,
f) gas feed/gas exhaust means in the form of a hollow central column (2, 2′),
wherein the inert bed (7, 7′, 7″) at least partially fills space between and adjacent to the hollow central column (2, 2′) and the active bed (6, 6′).

2. A reactor according to claim 1, wherein the active bed comprises an inner entry or exit area of a predetermined height and the inert bed (7, 7′, 7″) extends over at least 10% of said predetermined height of the inner entry or exit.

3. A reactor according to claim 2, wherein the inert bed extends over the entire predetermined height of the inner entry or exit area of the active bed (6,6′).

4. A reactor according to claim 1, wherein the inert bed (7, 7′, 7″) comprises metallic and/or ceramic packings.

5. A reactor according to claim 4, wherein the ceramic packings are Pall and/or Raschig rings.

6. A reactor according to claim 5, wherein the Pall and/or Raschig rings have a diameter of between 10 and 50 mm.

7. A reactor according to claim 1, wherein the inert bed (7, 7′, 7″) comprises a layer thickness of inert particles between 5 and 50%, of the layer thickness of the active bed (6, 6′).

8. A reactor according to claim 1, wherein the layer thickness of the inert bed (7, 7′, 7″) is between 5 and 50%, of the layer thickness of the active bed (6, 6′).

9. A reactor according to claim 1, wherein the gas-permeable and/or liquid-permeable barrier (c, c′) comprises a perforated sheet.

10. A reactor for chemical reactions and/or adsorptive separation processes, comprising:
a) a jacket (1),
b) two essentially annular beds (6, 6′, 7, 7′, 7″),
c) one bed (6, 6′) consisting essentially of a chemically and/or adsorptively active material (active bed) and one bed (7, 7′, 7″) consisting essentially of a material that is inert with respect to the chemical reaction that is to be carried out in the reactor (inert bed), said one bed (6,6′) surrounding said one bed (7,7′,7″),
d) gas feed/gas exhaust space (4) between the jacket (1) and the active bed (6, 6′) or optionally between the jacket (1) and an inert bed surrounding the active bed,
e) gas feed/gas exhaust means in the form of a hollow central column (2, 2′),
wherein the inert bed (7, 7′, 7″) at least partially fills space between and adjacent to the hollow central column (2, 2′) and the active bed (6, 6′).

11. A reactor according to claim 1, wherein in addition to the inert bed (7, 7′, 7″) located between the central column (2, 2′) and the active bed (6, 6′), the reactor comprises another inert bed that surrounds the active bed (6, 6′).

12. A reactor according to claim 8, wherein the layer thickness of the inert bed (7, 7′, 7″) is between 10 and 20%, of the layer thickness of the active bed (6, 6′).

13. A reactor according to claim 10 wherein the layer thickness of the inert bed (7, 7′, 7″) is between 5 and 50%, of the layer thickness of the active bed (6, 6′).

14. A reactor according to claim 1, wherein the reactor is devoid of internals or other measures within the central hollow column or in a space between the jacket of the reactor and the active bed.

15. A reactor according to claim 11, wherein the reactor is devoid of internals or other measures within the central hollow column or in a space between the jacket of the reactor and the active bed.
